(19) United States
(12) Patent Application Publication
(10) Pub. No.: US 2025/0389660 A1
(43) Pub. Date: Dec. 25, 2025

(54) INCUBATION SYSTEM WITH INDIVIDUALIZED IMAGING OF VESSELS

(71) Applicant: Analog Devices, Inc., Wilmington, DE (US)

(72) Inventors: Max Marcel Shulaker, Cambridge, MA (US); Alicia Ann Coto, Boston, MA (US); Gramham E. McGrath, Boston, MA (US); Samuel H. Fuller, Wellesley, MA (US)

(21) Appl. No.: 18/753,792

(22) Filed: Jun. 25, 2024

(51) Int. Cl. G01N 21/64; G01N 35/00; G01N 35/10

(52) U.S. Cl. CPC G01N 21/6458; G01N 21/6486; G01N 35/1083; G01N 2035/00356

(57) ABSTRACT

An advanced incubation system is disclosed for monitoring biological specimens. The system can include an incubation chamber and multiple specimen drawers, each drawer housing a plurality of receptacles designed to hold biological specimen vessels. These drawers can slide between an open position, allowing access to the receptacles, and a closed position, where they can be sealed within the incubation chamber. The system is equipped with a series of imagers, and each can be aligned with a receptacle to capture high-resolution digital images of the specimens. Additionally, the system can include a plurality of illuminators that provide consistent lighting across the receptacles.

### BACKGROUND

Blood incubation can be used to help detect foreign bodies, such as bacteria or other microorganisms, within the bloodstream of a human patient. For example, a blood sample can be taken from the patient and incubated to determine whether an infection exists. During an incubation period, certain microorganism cultures can multiply within the blood, enabling several techniques to detect infections. For example, a noticeable change in the sample's properties can develop throughout incubation. The sample can be analyzed to help form a medical diagnosis of the patient.

Incubation periods of a blood sample can range between about four hours to multiple days, depending upon the type of microorganism and the ambient temperature. The sample can be maintained at approximately body temperature (about 37° C.), or the temperature of the sample can be established such as to enhance growth of a certain target foreign body.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A depicts an incubation system including a plurality of specimen drawers in a closed position.

FIG. 1B depicts an incubation system including a plurality of specimen drawers in an open position.

FIG. 2A depicts a specimen drawer in an open position, exposing a plurality of receptacles.

FIG. 2B depicts an individual receptacle occupied by a specimen vessel.

FIG. 2C depicts a specimen vessel for receiving a biological specimen.

FIG. 3A depicts a specimen drawer in an open position, exposing a plurality of receptacles beneath respective imagers.

FIG. 3B depicts a plurality of imagers arranged on a printed circuit board (PCB).

FIG. 3C depicts the PCB of FIG. 3B, including a plurality of image processors each for receiving image data from respective imagers.

FIG. 4 illustrates a method for monitoring an incubated biological specimen.

FIG. 5 illustrates generally an example of a block diagram of a machine.

### DETAILED DESCRIPTION

Certain biological specimen incubation techniques can involve one or more manual operations during or between incubation sessions. Intervening manually in the incubating of a specimen can present challenges, such as difficulty in obtaining repeatable results and possible operator-introduced errors. Further, an incubation technique can involve visual inspection of biological specimens, such as for signs of an infection or other growth therein. This can include observing for one or more signs of a change in color, clarity, size, or other characteristics of the specimen. Manual approaches can present a challenge in achieving such visual inspection without introducing contamination or operator-introduced errors, such as to determine whether a target foreign body is present within the biological specimen. For example, certain techniques involve physical removal of specimens from incubators for observation, which can disrupt the controlled environment and affect growth within the biological specimen. Furthermore, manual approaches to monitoring biological specimen can result in inconsistent data, e.g., between different technicians. Certain approaches to automated incubation can involve large and very costly systems, such as relying on massive processing power (e.g., larger than available via a consumer device) and requiring use of complicated mechanisms for moving components within an incubation chamber. For example, such approaches can leverage robotics and gantries to physically move specimens and imaging equipment with respect to each other in attempt to replace incubation and monitoring actions via a human technician. The present inventors have recognized the benefits of a more user-friendly, more reproducible, less operator-dependent, more sterile, and more cost-efficient technique for biological specimen incubation and analysis.

This document describes a specimen imaging unit integrating an automated imaging system within the controlled environment of an incubation chamber. Such an arrangement enables monitoring of specimens without the need for manual intervention or robotic manipulation of individual biological specimens, thereby reducing a risk of contamination or otherwise disrupting the specimens during the analysis process. The system can include an array of high-resolution imagers, each corresponding to an individual biological specimen vessel. The imagers can facilitate that each specimen is captured in high detail automatically, e.g., with a resolution of at least 100 pixels per inch (PPI). Such a level of detail in imaging can aid in analyzing of the specimens' characteristics as they develop over time, unlike certain other approaches including sensors configured to obtain “images” with very few (e.g., <10) pixels. A uniform illumination provided by the system's illuminators can ensure consistent of imaging across several different biological specimen vessels, despite different positioning of the vessels within the incubation chamber. For example, by ensuring that each specimen is evenly lit, the system can reduce a variability in image quality and improve a reliability of the data collected. The system's image processor and system controller can analyze the captured images, extracting optical data that includes a range of properties such as color, absorption, reflection, fluorescence, and scattering. The inclusion of multiple processors within the system, including at least two image processors and a system controller, can help mitigate a challenge of processing a large volume of imaging data. Such architecture allows for efficient and simultaneous handling of data from multiple imagers.

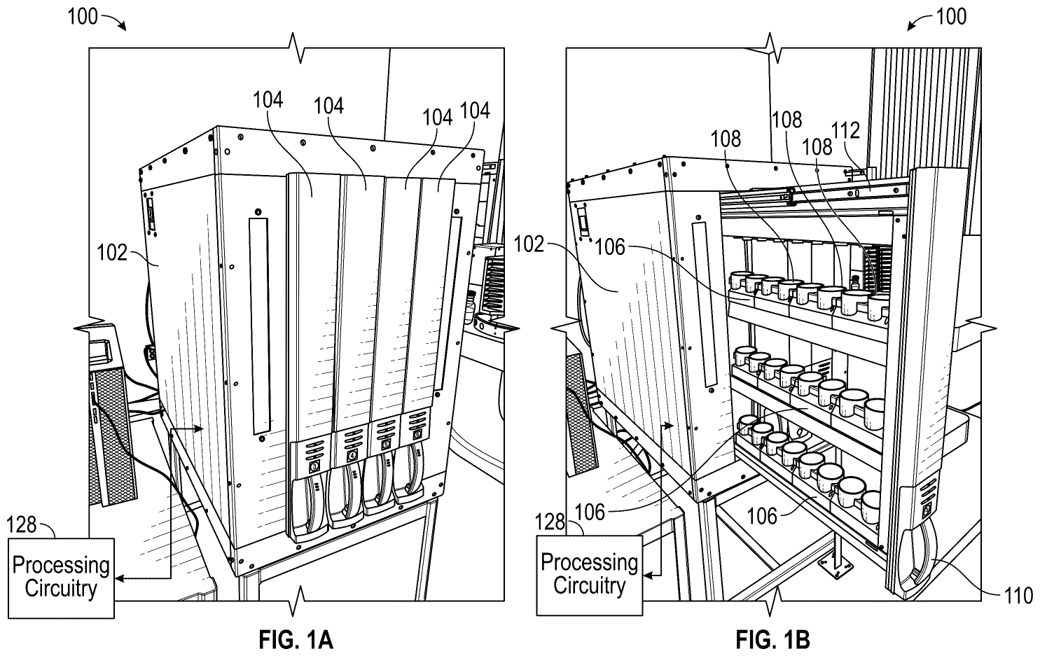

FIG. 1A and FIG. 1B depict an example of portions of an incubation system 100 including a plurality of specimen drawers 104 in open and closed positions, respectively. The incubation system 100 can include an incubation chamber 102 including a plurality of specimen drawers 104. The specimen drawers 104 can be arranged vertically within the incubation chamber 102, such that the drawers 104 stand side by side and rows 106 of an individual specimen drawer 104 are stacked atop one another when the incubation chamber 102 is in an upright orientation. In certain configurations (e.g., on drawer 104 in an open position as shown in FIG. 1B) such a vertical arrangement can facilitate similar access of each of the receptacles 108, each sized and shaped for holding an individual biological specimen vessel, from opposing sides of the same drawer 140. For example, such similar access of a receptacle 108 from opposing sides of the same drawer 104 can help prevent a need to reach over other receptacles 108 to access a desired receptacle 108. In another example, each of the specimen drawers 104 can be arranged horizontally (e.g., one atop the other) when the incubation chamber 102 is in the upright orientation. Here, the rows 106 can be arranged aside one another from a front side of a drawer 104 to a back side of the drawer 104. Where the specimen drawers 104 are arranged horizontally, the receptacles 108 can be exposed only via a single side of the drawer (e.g., a top side) unlike the depicted example where the drawers are arranged vertically and accessible from a plurality of opposing sides.

The specimen drawers 104 can be mounted to the incubation chamber 102, each via a sliding mechanism 112. The specimen drawers 104 can slide in and out (e.g., via operation of a handle 110) of the incubation chamber 102 along specified pathways corresponding to the sliding mechanism 112, which allows each of the specimen drawers 104 to slide independently of one another. Accordingly, sliding out a particular specimen drawer 104 exposes a subset of the receptacles 108 of that specimen drawer 104 from an otherwise enclosed environment within the incubation chamber 102. Accordingly, removal and subsequent opening of a specimen drawer 104 can allow efficient access to a particular row 106 of specimen receptacles 108, or access to each of the specimen receptacles 108 of multiple specimen drawers 104 without requiring a user to reach over other surrounding receptacles 108 or drawers 104. The incubation chamber 102 can be substantially enclosed, while each of the specimen drawers 104 are in a closed position, to isolate the specimen receptacles 108 within the enclosed environment for an intended maintenance and cultivation of biological specimens. For example, a seal or gasket can be arranged at an interface between the specimen drawers 104 and the incubation chamber 102. The seal or gasket can be formed of a resilient material (e.g., silicon rubber, etc.) to form a substantially air tight seal between the specimen drawer 104 and the incubation chamber 102 when the drawer 104 is closed.

In an example, at least one of an individual specimen drawer 104 or its corresponding sliding mechanism 112 can include a locking feature for securing an individual drawer 104 firmly in place in the extended position or in the closed position of the chamber 102. For example, the locking feature can be actuated via the handle 110 and can include a latch, gear, pressure spring, twist lock, hook and detent or any other similar mechanism able to adjustably secure a moveable member. Accordingly, the locking feature is able to hold the drawer partially extended from the incubation chamber 102 into an open position. Further, the locking feature can hold a drawer securely in a close position by preventing unintended movement of the drawer (e.g., movement due to vibration, shock or other event).

In an example, the incubation chamber 102 can further include or be communicatively coupled with processing circuitry 128 for controlling an environment of the incubation system 100. For example, the processing circuitry 128 can control a temperature, gas composition, lighting, humidity, or other environmental conditions associated with the incubation chamber 102 or other areas of the incubation system 100. The processing circuitry 128 can identify current conditions within the incubation chamber 102 and send instructions to one or more elements (e.g., a heater, a chiller, a circulator, a light source, a humidity producer, etc.) of the incubation system 100 to regulate environmental parameters within the enclosed environment. In an example, the processing circuitry 128 can receive data from one or more sensors (e.g., a transducer, a calibrated temperature sensing device, a calibrated pressure sensing device, a calibrated resistance sensor, a pH sensor, an oxygen sensing device, etc.) embedded within the incubation chamber 102 such as receiving data for monitoring temperatures or other environmental conditions. In an example, the processing circuitry can execute instructions provided via a local input/output (I/O) device or instructions provided via a network interface component (e.g., network interface card) coupled to the processing circuitry. For example, in response to a user manipulating the local I/O device, the processing circuitry can instruct e.g., a temperature regulator to raise or lower the incubation chamber temperature. In an example, the processing circuitry 128 can receive instructions over the network interface component to regulate or change conditions within the incubation chamber 102 from a remote user managing an incubation system 100, e.g., at or near the incubation chamber 102 or alternatively via a remote location.

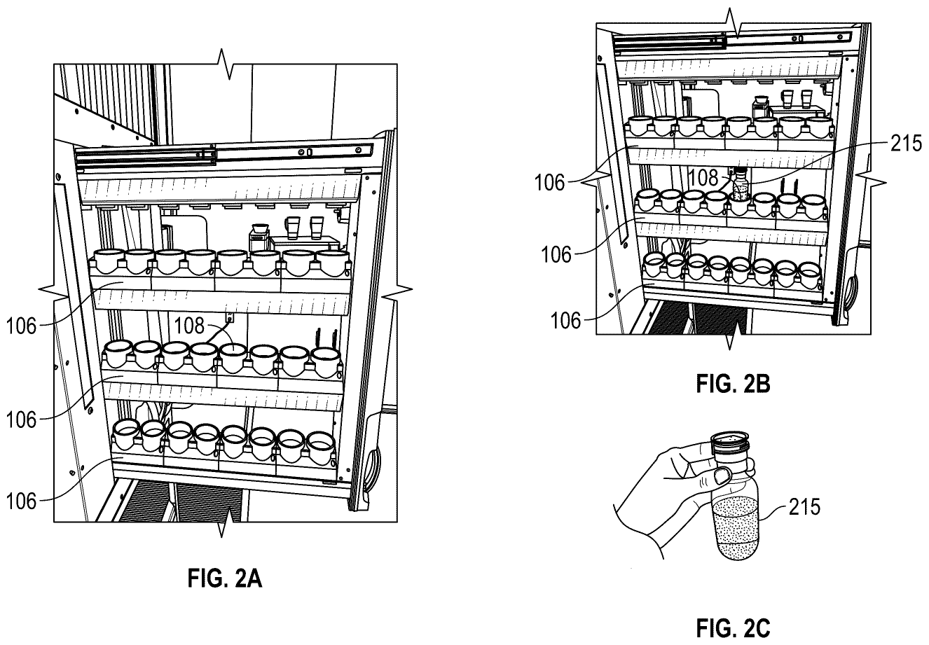

FIG. 2A and FIG. 2B depict an example of a specimen drawer 104 in an open position, exposing a plurality of receptacles 108. As shown in FIG. 2B, the receptacles 108 can be sized and shaped such as to accept an individual biological specimen vessel 215. Examples of a specimen vessel 215 can include, e.g., a vial, a Petri dish, a multiwell plate, a microtiter plate, a chip, a slide, a tube, such as a tube formed from a heat sealable plastic, a strip, a pad, or other suitable container that holds at least one biological specimen. For example, the biological specimen can include one or more of liquid, solid, colloidal, and gaseous components. The biological specimen can include one or more liquids such as blood, plasma, cerebrospinal fluid, synovial fluid, urine, sweat, saliva, transcutaneously obtained fluids (TTF), sputum, mucus, stool, gastric contents, and tissue.

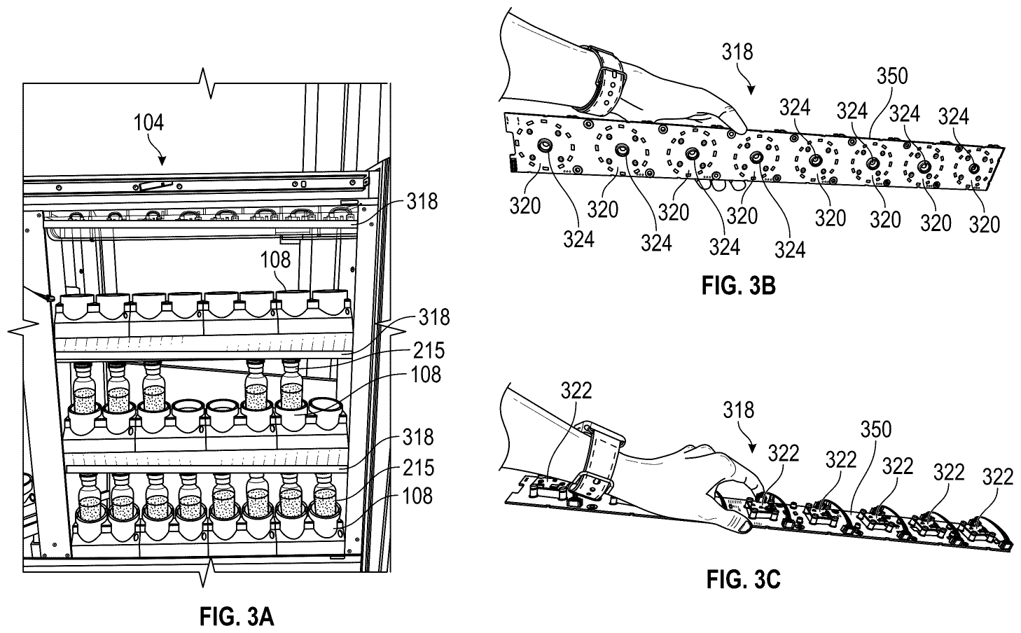

FIG. 2C shows an example of an individual specimen vessel. In an example, the specimen vessel 215 can include an openable, fluid-impermeable seal at a sealed aperture of the specimen container. For example, the fluid-impermeable seal can include a septum including, e.g., silicone, rubber, or plastic affixed (e.g., adhered or fastened) to the specimen vessel 215. The seal can provide a sterile environment within the specimen vessel 215, for example, by preventing contamination or inadvertent ingress of fluid, dust particles, or other bacteria into the specimen vessel 215. In an example, the seal can include a gas-permeable membrane, such as such as a polyethylene, polytetrafluoroethylene, or other type of thin film or membrane material. The gas-permeable membrane can include a liquid impervious property, such as to impede liquid flow therethrough. In an example, the seal can be perforable to establish a passageway to an interior of the specimen container, e.g., upon application of a force toward the membrane (e.g., a piercing or puncturing force). For example, a piercing element such as a syringe needle can be used to puncture the membrane. Upon removal of the syringe from the membrane, the membrane can return to its resting, sealed, or unperforated state (e.g., resealed). Such resealing can prevent leakage or escaping of the biological specimen from the specimen vessel 215. Alternatively or additionally, the specimen vessel 215 can include or be sized and shaped to accept (e.g., embedded within a cap for interfacing with the vessel) a seal perforator, and the seal perforator can permanently puncture the membrane to avoid resealing via the fluid-impermeable seal. Here, the seal perforator can be screwed or snapped to the specimen vessel 215 such as to establish a new seal, different than the fluid-impermeable seal, following the puncturing. In an example, the seal perforator can facilitate selective access to contents within the specimen vessel 215 and resealing via the new seal. In an example, the specimen vessel 215 can include or use a sensor embedded in at least one of the specimen vessel 215 or the seal perforator, e.g., to monitor for growth or condition of the biological specimen. For example, the specimen vessel 215 can include an onboard electrochemical transducer incorporated within the vessel. Thus, the specimen vessel 215 can be accessed for individual electrochemical transduction, such as gas sensing, e.g., concurrent with an imaging of the specimen vessel 215 via an imager (e.g., the imagers 318 as depicted in FIG. 3A, FIG. 3B, and FIG. 3C). In an example, the onboard electrochemical transducer can include transceiver circuitry to transmit the electrical response signal to a location outside the incubation chamber 102. Alternatively or additionally, the specimen vessel 215 can include a port sized and shaped such as to be communicatively coupled with a corresponding receptacle 108. Here, the receptacle 108 can include or use an electrochemical transducer to transduce, via the port, an electrical property, indicative of a target gas composition corresponding with the particular biological specimen, into an electrical response signal. The specimen vessel 215 can similarly be connected, e.g., via the port, to an optical sensor for obtaining an optical property indicative of a target gas composition corresponding with the particular biological specimen.

FIG. 3A depicts an example of a specimen drawer 104 in an open position, exposing a plurality of receptacles 108 beneath respective imagers 318. FIG. 3B depicts a plurality of imagers 318 arranged on a printed circuit board (PCB) 350. FIG. 3C depicts the PCB of FIG. 3B, including a plurality of image processors 322 each for receiving image data from respective imagers 318.

In an example, the specimen drawer 104 can include a plurality of receptacles 108, each sized and shaped to receive a biological specimen vessel 215. The specimen drawer 104 can also include one or more imagers 318, and an individual imager 318 can include a PCB 350 including a plurality of illuminators 320, a plurality of image processors 322, and a plurality of sensors 324.

In an example, system 100 (as shown in FIG. 1A and FIG. 1B) can facilitate monitoring and analysis of individual biological specimen vessels 215, each via a corresponding imager 318, e.g., the corresponding imager 318 aligned with a corresponding receptacle 108. In an example, the imagers 318 can each be capable of producing a digital image with a resolution of at least 100 pixels per inch (PPI), e.g., via an array 324 of detectors.

The system can also include a plurality of illuminators 320, e.g., a broadband or tunable wavelength electromagnetic energy source, that collectively provide uniform illumination to each specimen vessel 215 received in the specimen drawer 104, e.g., capable of providing uniform illumination to each receptacle 108 in the specimen drawer 104. Herein, “uniform illumination” refers to illumination that is approximately equal in one or both of intensity and spectral emission distribution across each receptacle 108 and each specimen vessel 215 received therein, such as varying (in average intensity or spectral emission distribution) from vessel to vessel no more than 20%, no more than 10%, or no more than 5%. The uniform illumination may also be substantially consistent across the vessel length or width, such as measured to within 20%, 10%, or 5% of the average intensity or spectral emission distribution across the longest or shortest dimension of the vessel. In an example, an individual illuminator 320 includes an array of emitters arranged (e.g., in a ring shape, a linear array, or an angular arrangement of at least two linear arrays) to distribute light evenly across an area greater than 1 square centimeter (cm²). The emitters can include, e.g., light emitting diodes (LEDs), laser diodes, or an organic light emitting diode (OLED) panel.

In an example, one or more image processors 322 can commence individual imaging of respective specimen vessel 215, while separate a system controller (e.g., the processing circuitry 128 as depicted in FIG. 1A and FIG. 1B) can receive and analyze digital images from the one or more image processors 322. In an example, the system controller can extract optical data such as color, absorption, reflection, fluorescence, or scattering from the received digital images. The system controller can also characterize the biological specimen within the specimen vessels 215 via data analysis. For example, the system controller can calculate probabilities for identifying specific characteristics of the specimens, such as detecting infectious microorganisms or determining their growth properties and antibiotic susceptibility.

The imager 318 can include an array 324 of detectors. The array 324 can be used to generate a digital representation of an individual biological specimen vessel 215. For example, the digital representation can be processed to generate image data for further storage or display. The specimen image can be processed and analyzed to provide quantitative information on the biological material in the specimen vessel 215. For example, the processing and analysis can include one or more of determining cell or pathogen counts, cell or nucleus morphometry, cell size measurements, growth rate measurements, or other suitable specimen monitoring techniques of the biological specimen. In an example, the array 324 can comprise photosensitive elements configured to detect electromagnetic energy within the specified wavelength band, e.g., visible light or the near-infrared spectrum. Alternatively or additionally, the array 324 can include a solid-state imaging array of detector pixels configured for detecting specific scattering, fluorescence emissions, or other optical signatures from the target biological specimen. In an example, the array 324 can include or use a photodetector, charged-coupled-device (CCD), complementary metal-oxide-semiconductor (CMOS) detector, or array camera, e.g., operably coupled to the PCB 350 to receive and record light energy imaged from a specimen vessel 215.

In an example, an individual image processor 322, including the plurality of arrays 324 and corresponding illuminators 320, can be arranged on the PCB 350. The PCB 350 can include a board or substrate, e.g., a ceramic or polymeric circuit board, mounting, connecting, and housing of electrical components, e.g., as one or more integrated circuits (ICs).

In an example, The PCB 350 includes a plurality of image processors 322, and an individual image processor 322 can include, e.g., a Field Programmable Gate Array (FPGA), Digital Signal Processor (DSP), or other type of programmable logic for processing data from a corresponding array 324 to provide a digital representation of the specimen in the biological specimen vessel 215. In an example, the individual image processor 322 is clocked by the system clock on the PCB, and the processor 322 be configured to process received frames or signals from the array 324 and to send digital versions of the image frames to the image storage or analysis unit. The individual image processor can also include instructions to perform operations of an image processing algorithm, e.g., residing on the image analysis unit, for analyzing the images from the illumination (e.g., via the corresponding illuminator 320) reflected from the biological specimen in the specimen vessel and detect scattering, absorption, fluorescence, or other phenotypic signals.

Ultimately, the image processing algorithm can characterize a specified target biological specimen carried by a corresponding biological specimen vessel 215. For example, the image processing algorithm can calculate respective probabilities of subsequent identification of the specified target biological specimen for a corresponding biological specimen vessel 215. The identification can include, e.g., detection of an infectious microorganism or identification of growth properties, antibiotic susceptibility, genus, species, or strain of the microorganism.

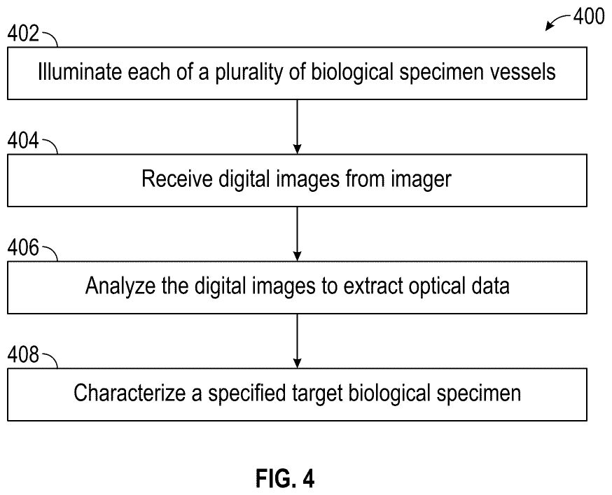

FIG. 4 is a flowchart describing an example of portions of a process of monitoring an incubated biological specimen via an incubation system. For example, the process can be performed using the system 100 of FIG. 1A, e.g., including performing operations on one or more of the processing circuitry 128 or one or more of the image processors 322 of FIG. 3A, FIG. 3B, and FIG. 3C.

At 402, the process can commence with a uniform illumination of biological specimen vessels arranged within a sealed incubation chamber. The illumination can be provided by one or more illuminators, an individual illuminator, e.g., comprising an arrangement of emitters for delivering substantially even illumination across a defined area. Such uniform illumination can promote that each specimen vessel is lit consistently for optimal image capture, such as for comparable imaging results from vessel to vessel.

At 404, digital images of the specimen vessels can be captured via one or more imagers, and the one or more imagers can include detectors being individually associated with a corresponding specimen vessel. In an example, the one or more imagers can include arrays of detectors, e.g., greater than 100×100 individual detectors, to produce digital images with a resolution of at least 100 pixels per inch (PPI). This relatively high-resolution (e.g., not binary imaging) imaging capability can facilitate a capture of intricate details to assist in thorough specimen analysis.

At 406, images can be processed to extract optical data. The data can include or exhibit various optical properties, such as color, absorption, reflection, fluorescence, elastic scattering, or inelastic (Raman) scattering. The processing and analysis of this data can be facilitated by an image processor, which may be one of multiple processors within the system, including at least two image processors configured to handle the imaging data efficiently.

At 408, a specified target biological specimen can be characterized. For example, a system controller, which can be a separate processor or integrated with the image processor, can receive the imaging data and can facilitate a detailed analysis to characterize the specimen. For example, the system controller can facilitate a calculation of probabilities for subsequent identification of the specimen, which can involve detecting infectious microorganisms or identifying their growth properties, antibiotic susceptibility, and taxonomic classification down to the genus, species, or strain level.

In an example, one or more biological specimen drawers, housing the biological specimen vessels, can be mechanically manipulated. An individual drawer, defining multiple receptacles for the specimen vessels, can be capable of sliding between an open position, where the drawer extends from the incubation chamber to expose the receptacles to an external ambient environment, and a closed position, where the drawer is retracted into the chamber, sealing the receptacles within. Such movement can facilitate a placement of the specimen vessels into the system and a sealing of an incubation chamber, e.g., for maintenance of a controlled environment during incubation and imaging.

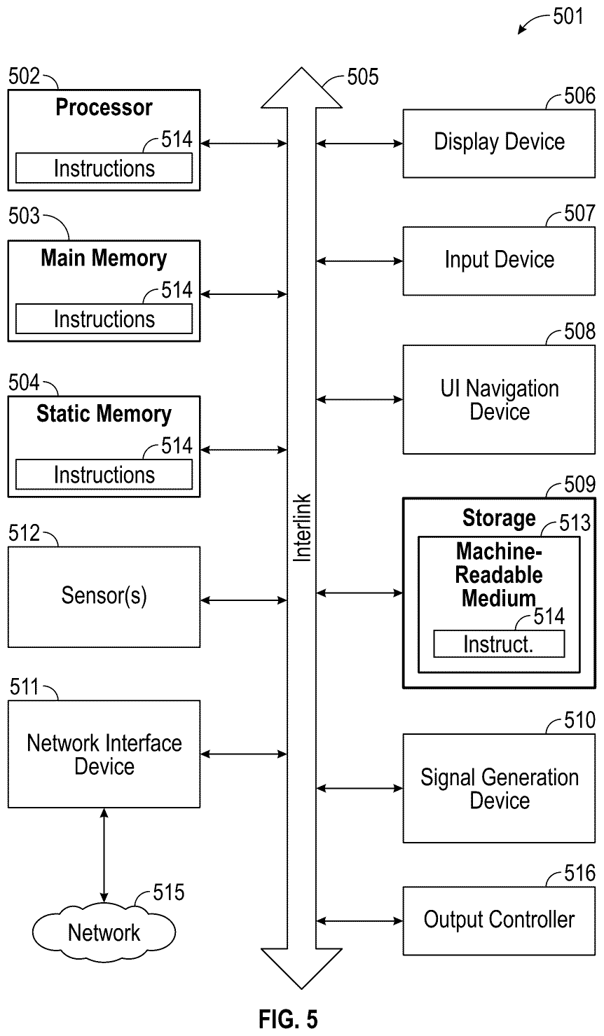

FIG. 5 illustrates generally an example of a block diagram of portions of a machine 501 upon which any one or more of the techniques (e.g., methodologies) discussed herein may perform in accordance with some examples. In alternative embodiments, the machine 501 may operate as a standalone device or may be connected (e.g., networked) to other machines. In a networked deployment, the machine 501 may operate in the capacity of a server machine, a client machine, or both in server-client network environments, or as a virtual machine. In an example, the machine 501 may act as a peer machine in peer-to-peer (P2P) (or other distributed) network environment. The machine 501 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (Saas), other computer cluster configurations.

Examples, as described herein, may include, or may operate on, logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In an example, the hardware may include configurable execution units (e.g., transistors, circuits, etc.) and a computer readable medium containing instructions, where the instructions configure the execution units to carry out a specific operation when in operation. The configuring may occur under the direction of the executions units or a loading mechanism. Accordingly, the execution units are communicatively coupled to the computer readable medium when the device is operating. In this example, the execution units may be a member of more than one module. For example, under operation, the execution units may be configured by a first set of instructions to implement a first module at one point in time and reconfigured by a second set of instructions to implement a second module.

Machine (e.g., computer system) machine 501 may include a hardware processor 502 (e.g., a central processing unit (CPU), a graphics processing unit (GPU), a hardware processor core, or any combination thereof), a main memory 503 and a static memory 504, some or all of which may communicate with each other via an interlink (e.g., bus) 505. The machine 501 may further include a display unit 506, an alphanumeric input device 507 (e.g., a keyboard), and a user interface (UI) navigation device 508 (e.g., a mouse). In an example, the display unit 506, alphanumeric input device 507 and UI navigation device 508 may be a touch screen display. The machine 501 may additionally include a storage device (e.g., drive unit) 509, a signal generation device 510 (e.g., a speaker), a network interface device 511, and one or more sensors 512, such as a global positioning system (GPS) sensor, compass, accelerometer, or other sensor. The machine 501 may include an output controller 516, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 509 may include a machine readable medium 513 that is non-transitory on which is stored one or more sets of data structures or instructions 514 (e.g., software) embodying or utilized by any one or more of the techniques or functions described herein. The instructions 514 may also reside, completely or at least partially, within the main memory 503, within static memory 504, or within the hardware processor 502 during execution thereof by the machine 501. In an example, one or any combination of the hardware processor 502, the main memory 503, the static memory 504, or the storage device 509 may constitute machine readable media.

While the machine readable medium 513 is illustrated as a single medium, the term “machine readable medium” may include a single medium or multiple media (e.g., a centralized or distributed database, or associated caches and servers) configured to store the one or more instructions 514.

The term “machine readable medium” may include any medium that is capable of storing, encoding, or carrying instructions for execution by the machine 501 and that cause the machine 501 to perform any one or more of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 514 may further be transmitted or received over a communications network 515 using a transmission medium via the network interface device 511 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 502.11 family of standards known as Wi-Fi®, IEEE 502.16 family of standards known as WiMax®), IEEE 502.15.4 family of standards, peer-to-peer (P2P) networks, among others. In an example, the network interface device 511 may include one or more physical jacks (e.g., Ethernet, coaxial, or phone jacks) or one or more antennas to connect to the communications network 515. In an example, the network interface device 511 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 501, and includes digital or analog communications signals or other intangible medium to facilitate communication of such software.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An incubation system for monitoring an incubated biological specimen, the system comprising:
an incubation chamber;
one or more specimen drawers each defining a plurality of receptacles, an individual receptacle sized and shaped for receiving a corresponding biological specimen vessel, wherein an individual specimen drawer of the one or more specimen drawers is configured to slide toward a closed position wherein the one or more specimen drawers is withdrawn into the incubation chamber and the plurality of receptacles are contained within the chamber and sealed from an external ambient environment;
a plurality of imagers, each corresponding with an individual receptacle of the plurality of receptacles and each configured to produce a digital image of a respective specimen vessel arranged in a corresponding receptacle, wherein the digital image includes a plurality of pixels;
one or more illuminators collectively arranged to provide like illumination to each of the plurality of receptacles of the one or more specimen drawers; and
an image processor configured to commence imaging via one or more imagers of the plurality of imagers.

2. The incubation system of claim 1, wherein the incubation system is configured to alternate between:
an open position at least partially exposing the plurality of receptacles to be loaded or unloaded with specimen vessels; and
the closed position, wherein the plurality of receptacles are contained within the chamber and sealed from the external ambient environment.

3. The incubation system of claim 1, comprising a system controller configured to:
receive digital images, each corresponding with a respective receptacle;
analyze the digital images to extract optical data corresponding with at least one of color, absorption, reflection, fluorescence, elastic scattering, or inelastic (Raman) scattering; and
characterize a specified target biological specimen carried by a corresponding biological specimen vessel.

4. The incubation system of claim 3, comprising a system controller configured to receive imaging data from the image processor.

5. The incubation system of claim 3, comprising first and second processors, including the image processor, and further comprising a third processor including a system controller configured to receive imaging data from each of the first and second processors.

6. The incubation system of claim 3, wherein the system controller is further configured to calculate respective probabilities of subsequent identification of the specified target biological specimen for a corresponding biological specimen vessel, the subsequent identification including at least one of:
detection of an infectious microorganism; or
identification of growth properties, antibiotic susceptibility, genus, species, or strain of the microorganism.

7. The incubation system of claim 1, wherein the digital image includes resolution of at least 100 pixels per inch (PPI).

8. The incubation system of claim 7, wherein each of the plurality of imagers includes an array of detectors, wherein the array is greater than 100×100 individual detectors and each individual detector corresponds to an individual pixel of the at least 100 PPI.

9. The incubation system of claim 1, wherein the one or more illuminators include a plurality of illuminators, respectively corresponding with an individual receptacle of the plurality of receptacles.

10. The incubation system of claim 8, wherein an individual one of the plurality of illuminators includes a ring of emitters configured to provide substantially even illumination across an area greater than 1 square centimeter (cm²).

11. An incubation system for monitoring an incubated biological specimen, the system comprising:
an incubation chamber;
a plurality of specimen drawers, each specimen drawer defining a plurality of receptacles, an individual receptacle sized and shaped for receiving a corresponding biological specimen vessel, wherein each specimen drawer of the plurality of specimen drawers is independently configured to slide toward a closed position wherein each of the plurality of specimen drawers is withdrawn into the incubation chamber and the plurality of receptacles are contained within the chamber and sealed from an external ambient environment;
a plurality of imagers each including an array of detectors, each imager corresponding with an individual receptacle of the plurality of receptacles and each configured to produce a digital image of a respective specimen vessel arranged in a corresponding receptacle, wherein each array comprises greater than 100×100 individual detectors;
one or more illuminators collectively arranged to provide like illumination to each of the plurality of receptacles of the one or more specimen drawers; and
at least two image processors configured to commence imaging via one or more imagers of the plurality of imagers; and
a system controller configured to receive imaging data from each of the at least two image processors.

12. The incubation system of claim 11, wherein the incubation system is configured to alternate between:
an open position at least partially exposing the plurality of receptacles to be loaded or unloaded with specimen vessels; and
the closed position, wherein the plurality of receptacles are contained within the chamber and sealed from the external ambient environment.

13. The incubation system of claim 11, comprising a system controller configured to:
receive digital images, each corresponding with a respective receptacle;
analyze the digital images to extract optical data corresponding with at least one of color, absorption, reflection, fluorescence, elastic scattering, or inelastic (Raman) scattering; and
characterize a specified target biological specimen carried by a corresponding biological specimen vessel.

14. The incubation system of claim 13, wherein the system controller is further configured to calculate respective probabilities of subsequent identification of the specified target biological specimen for a corresponding biological specimen vessel, the subsequent identification including at least one of:
detection of an infectious microorganism; or
identification of growth properties, antibiotic susceptibility, genus, species, or strain of the microorganism.

15. The incubation system of claim 11, wherein the one or more illuminators include a plurality of illuminators, respectively corresponding with an individual receptacle of the plurality of receptacles, wherein an individual one of the plurality of illuminators includes a ring of emitters configured to provide substantially even illumination across an area greater than 1 square centimeter (cm²).

16. A method for monitoring an incubated biological specimen, the method comprising:
illuminating, via one or more illuminators, each of a plurality of biological specimen vessels, arranged in a sealed incubation chamber, at a same illumination;
receiving respective digital images from a plurality of imagers, each imager individually corresponding with one of the plurality of biological specimen vessels arranged in a sealed incubation chamber and each imager configured to produce a digital image of a respective specimen vessel, wherein the digital image includes a plurality of pixels;
analyzing the digital images to extract optical data corresponding with at least one of color, absorption, reflection, fluorescence, elastic scattering, or inelastic (Raman) scattering; and
characterizing a specified target biological specimen carried by a corresponding biological specimen vessel.

17. The method of claim 16, comprising:
moving one or more specimen drawers, each defining a plurality of receptacles with each receptacle sized and shaped for receiving a corresponding biological specimen vessel, between:
an open position wherein an individual specimen drawer extends from the incubation chamber to expose the plurality of receptacles to an external ambient environment; and
a closed position wherein each the one or more specimen drawers is withdrawn into the incubation chamber and the plurality of receptacles are contained within the chamber and sealed from the external ambient environment.

18. The method of claim 16, comprising calculating respective probabilities of subsequent identification of the specified target biological specimen for a corresponding biological specimen vessel, the subsequent identification including at least one of:
detection of an infectious microorganism; or
identification of growth properties, antibiotic susceptibility, genus, species, or strain of the microorganism.

19. The method of claim 16, wherein the digital image includes resolution of at least 100 pixels per inch (PPI).

20. The method of claim 19, wherein each of the plurality of imagers includes an array of detectors, wherein the array is greater than 100×100 individual detectors and each individual detector corresponds to an individual pixel of the at least 100 PPI.
